(19) United States
(12) Patent Application Publication
(10) Pub. No.: US 2013/0316002 A1
(43) Pub. Date: Nov. 28, 2013

(54) COMPOSITIONS AND METHODS FOR TREATING GASTROINTESTINAL MOTILITY DYSFUNCTION

(71) Applicant: Depomed, Inc., Newark, CA (US)

(72) Inventor: Bret Berner, Half Moon Bay, CA (US)

(73) Assignee: Depomed, Inc., Newark, CA (US)

(21) Appl. No.: 13/900,312

(22) Filed: May 22, 2013

Related U.S. Application Data
(60) Provisional application No. 61/650,451, filed on May 22, 2012.

(51) Int. Cl. A61K 9/20; A61K 38/02

(52) U.S. Cl. CPC A61K 9/2031; A61K 38/02
USPC 424/490; 514/9.7

(57) ABSTRACT

Extended release oral dosage forms for the treatment of gastrointestinal motility disorders are described. Active agents which are small molecules, peptides, peptide analogs, and peptide mimetics are formulated for optimal release in the GI tract of subjects with GI motility dysfunction. Methods of treatment using the dosage forms are also described.

### CROSS-REFERENCE TO RELATED APPLICATIONS

This application dams the benefit of U.S. Provisional Application No. 61/650,451, filed on May 22, 2012, the contents of which are incorporated herein by reference in its entirety.

### TECHNICAL FIELD

Extended release oral dosage forms which contain active agents for the treatment of gastrointestinal motility disorders and methods of use for the treatment of gastrointestinal motility disorders are described. The dosage forms provide extended release of an active agent to the gastrointestinal tract of a subject.

### BACKGROUND

Disorders of gastrointestinal motility include, for example, gastroparesis and gastroesophageal reflux disease (GERD). The impairment of gastrointestinal motility may result in a variety of other ailments including irritable bowel syndrome (IBS), constipation (e.g. that associated with the hypomotility phase of IBS), emesis (e.g., that caused by cancer chemotherapy agents), ileus and colonic pseudo-obstruction, anorexia, gall bladder stasis, gastritis, chronic constipation (colonic inertia), and dyspepsia.

Gastroparesis is the delayed emptying of stomach contents brought about by a motor abnormality in the stomach, as a complication of diseases such as diabetes, progressive systemic sclerosis, anorexia nervosa, or myotonic dystrophy. Acute gastroparesis may be caused by, for example, surgery (postoperative paralytic ileus), drugs (e.g., opioids), viral enteritis, and hyperglycemia, and is usually managed by treating the underlying disease rather than the motility disorder. The most common causes of chronic gastroparesis are associated with long standing diabetes or idiopathic pseudo-obstruction, often with so-called “non-ulcer” or “functional” dyspepsia.

These GI disorders are generally treated with prokinetic agents that enhance GI motility, One class of prokinetic agents is 5-HT4 receptor modulators, which stimulate gut motility by modulating the ability of 5-hydroxytryptamine (5-HT, i.e. serotonin) to stimulate gut motility. This class of agents includes metoclopramide, cisapride, and other benzamide derivatives. Dopamine receptor antagonists, for example doperidome and itopride, are another class of agents that possess GI prokinetic activity. Many of these agents possess antiemetic activity as well as prokinetic activity. Still another class of prokinetic agents is motilin receptor modulators, including macrolide compounds such as erythromycin and its derivatives that are agonists of the motilin receptor, as well as the peptide motilin and motilin peptide analogs. Yet another class of prokinetic agents are growth hormone secretagogue receptor modulators.

Peptides affecting the release of growth hormone (GH) are thought to exhibit gastrokinetic or “prokinetic” effects (U.S. Pat. No. 6,548,501; Peeters, T. L., J. Physiol. Pharmacol., (2003), 54 (supp 4):95-103 and references therein; Trudel, L. et al, J. Physiol. Gastrointest. Liver Physiol., (2002), 282:G948-52), Such growth hormone-releasing peptides, or GHRPs, are also referred to as growth hormone secretagogues (GHS). Exemplary growth hormone-releasing peptides (GHRPs) believed to exhibit prokinetic effects include GHRP-1, GHRP-2 and ghrelin. Ghrelin is produced by epithelial cells lining the fundus of the stomach and functions to stimulate appetite; its levels increase prior to a meal and decrease thereafter.

Oral delivery of these prokinetic agents is the preferred route of administration. For patients with gastroparesis, however, drug absorption through the GI tract is often unpredictable and far less effective than intravenous administration. Another limitation in oral delivery of these prokinetic agents is that some of these agents are peptides. Bioavailability of peptides is generally less than 1% due to poor absorption and instability, both enzymatic and hydrolytic, in the stomach. Thus, it is difficult to achieve efficacious plasma concentrations of orally administered peptides. There remains a need for oral delivery dosage forms for drugs for the treatment of GI motility disorders. Particularly, ER extended release (ER) oral dosage forms which can increase the retention time of the dosage form in the upper GI tract, and provide continuous, controlled delivery are needed, especially for drugs which have relatively low solubility in the acidic environment of the stomach and/or are unstable to enzymes and acid in the stomach.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

This disclosure is directed to methods to prevent, treat or ameliorate a gastrointestinal motility disorder using oral extended release (ER) dosage forms. Compositions for ER dosage forms to treat a gastrointestinal (GI) motility disorder are also provided. The administration of an ER oral dosage form containing an active agent for the treatment of a GI motility disorder to a subject with GI motility dysfunction may decrease the severity of the symptoms of the disorder. For some conditions, this treatment may also lead to disease resolution and/or prevent or reduce disease progression. The dosage forms may be used to deliver active agents for local treatment in the gastrointestinal tract as well as for systemic treatment, In certain embodiments, additional active agents may be administered, either separately or together with the ER dosage form containing the active agent for treating a GI motility disorder.

The compositions described herein for treating a GI motility disorder include oral dosage forms comprising an active agent which is effective for treating a subject suffering from or diagnosed with the GI motility disorder. Among the various aspects of the present disclosure, the compositions include extended release dosage forms.

In one aspect, an ER oral dosage form comprising a therapeutically effective amount of an active agent for treating a subject suffering from or diagnosed with a GI motility disorder is provided.

In one embodiment, the active agent is a small molecule, a peptide, a peptide analog or a peptide mimetic.

In one embodiment, the active agent is a motilin receptor modulator, a 5-HT4 receptor modulator, a dopamine receptor antagonist, a growth hormone secretagogue receptor modulator, an acetylcholinesterase inhibitor, a muscarinic receptor agonist, or a cholecystokinin receptor antagonist.

In one embodiment, the ER oral dosage form releases the active agent over a period of about 5 to 15 hours, 6 to 12 hours or 8 to 10 hours.

In one embodiment, the ER oral dosage form is a tablet or capsule. In another embodiment, the ER dosage form is a bilayer tablet, wherein one or both layers comprise the active agent. In still another embodiment, the ER dosage form is a multilayer tablet, wherein the one or more layers comprise the active agent. In yet another embodiment, the one layer or more layers of the bilayer or multilayer tablet comprise a second active agent.

In one embodiment, the ER dosage form is a gastric retained (GR) dosage form. In one embodiment, the gastric retained dosage form provides extended release of the active agent into the stomach, duodenum, and small intestine. In another embodiment, the extended release occurs over a period of about 4 to 16 hours, 6 to 12 hours or 5 to 10 hours.

In one embodiment, the gastric retained dosage form may contain an additional active agent.

In one aspect, the gastric retained dosage form is a tablet or a capsule. In another embodiment, the gastric retained dosage form is a bilayer tablet, wherein one or both layers comprise the active agent. In still another embodiment, the gastric retained dosage form is a multilayer tablet, wherein one or more layers comprise the active agent. In yet another embodiment, the one layer or more layers of the bilayer or multilayer tablet comprise an additional active agent.

In one embodiment, the gastric retained dosage form comprises the active agent and a swellable polymer. In another embodiment, the swellable polymer is a polyethylene oxide. In another embodiment, the swellable polymer is an alkyl substituted cellulose, preferably hydroxypropylmethylcellulose, carboxymethylcellulose, hydroxyethylcellulose, or hydroxypropylcellulose. Still another embodiment comprises a combination of two swellable polymers comprising polyethylene oxide and/or hydroxypropylmethylcellulose. In one embodiment, the swellable polymer forms a matrix that swells unrestrained dimensionally when contacted with a fluid, such as gastric fluid. In another embodiment, the swelling matrix is in the form of a single, monolithic matrix comprising one or both of the active agents dispersed therein.

In one embodiment, the gastric retained dosage form swells after administration to a subject to a size sufficient to promote gastric retention in the fed stomach.

In one embodiment, the ER dosage form is formulated to a size sufficient to promote retention in a stomach in the fed mode, In another embodiment, the total weight of the ER dosage form is about 750 mg to 1250 mg.

In one embodiment, the ER dosage form is an osmotic dosage form formulated to be a size sufficient to promote retention in a stomach in the fed mode.

In one embodiment, the ER dosage form comprises a buffering agent. In another embodiment, the ER dosage form comprises an antioxidant. In yet another embodiment, the ER dosage form comprises a buffering agent and an antioxidant.

In one embodiment, the ER dosage form comprises particles, wherein the particles comprise the active agent and wherein the particles are dispersed in the dosage form. In another embodiment, the particles are enteric-coated particles.

In one embodiment, the ER dosage form releases the active agent via diffusion. In another embodiment, the ER dosage form releases the active agent via erosion. In yet another embodiment, the ER dosage form releases the active agent via a combination of diffusion and erosion.

In another aspect, the disclosure relates to a method for treating a GI motility disorder comprising administering to a mammal a therapeutically effective amount of an active agent wherein said disorder is selected from GI motility disorders including, but not limited to, irritable bowel syndrome, constipation, gastroparesis, GERD, emesis, ileus, gastritis, and dyspepsia.

In one embodiment, a method for treating gastroparesis comprising administering to a mammal a therapeutically effective amount of an active agent in an ER oral dosage form is provided.

In one embodiment, the active agent is selected from a small molecule, a peptide, a peptide analog or a peptide mimetic. In another embodiment, the active agent is a motilin receptor modulator, a 5-HT4 receptor modulator, a dopamine receptor antagonist, or a growth hormone secretagogue receptor modulator.

In one embodiment, the second active agent is selected from a small molecule, a peptide, a peptide analog or a peptide mimetic. In another embodiment, the second active agent is a motilin receptor modulator, a 5-HT4 receptor modulator, a dopamine receptor antagonist, or a growth hormone secretagogue receptor modulator,

In one embodiment, the method reduces one or more symptoms associated with the disorder.

An effective dose of the active agent for the treatment of GI motility disorders with oral ER dosage forms will vary depending on factors including the bioavailability and the potency of the drug. The dosing frequency will depend on the total daily dose and the type of dosage form. For ER dosage forms, dosing will typically be once- or twice-daily. For gastric retentive dosage forms, dosing will typically be once- or twice-daily, with or without a meal.

For gastric retentive dosage forms, the increased motility of the stomach resulting from the administration of an active agent for the treatment of a GI motility disorder increases emptying from the stomach. Thus, administration of an agent to treat gastroparesis from a gastric retained dosage form would promote the dosage form to leave the stomach and cease the efficacious delivery of the drug. In spite of this deleterious effect on retention in the stomach, efficacious once- or twice-daily treatment of gastroparesis is described.

### DETAILED DESCRIPTION

Methods of treating and/or preventing gastrointestinal motility disorders using extended release oral compositions are provided. The oral administration of an active agent for the treatment of a gastrointestinal disorder using an ER dosage form provides for improvement or therapeutic treatment of abnormal gastrointestinal motility function. The ER oral dosage forms provide benefits over immediate release (IR) dosage forms such as decreased dosing frequency and improved bioavailability. The dosage forms of the present disclosure provide continuous and controlled delivery of an active agent to the GI tract of a subject suffering from a GI motility disorder. In some embodiments, the extended release dosage form is a gastric retained dosage form which delivers an active agent which stimulates gastric motility.

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art.

### Definitions

It must be noted that as used in this specification and the appended claims, the singular forms “a,” “an” and “the” include plural referents unless the context clearly dictates otherwise, Thus, for example, reference to “an active agent” or “a pharmacologically active agent” includes a single active agent as well a two or more different active agents in combination, reference to “a polymer” includes mixtures of two or more polymers as well as a single polymer, and the like.

“Preventing,” in reference to a disorder or unwanted physiological event in a patient, refers specifically to inhibiting or reducing the occurrence of symptoms associated with the disorder and/or the underlying cause of the symptoms.

“Ameliorate,” in reference to a disorder or unwanted physiological event in a patient, refers to improving or making better the occurrence of symptoms associated with the disorder and/or the underlying cause of the symptoms.

The term “treating”, as used herein, refers to reversing, alleviating, ameliorating, inhibiting the progress of, or preventing the disorder or condition to which such term applies, or preventing one or more symptoms of such condition or disorder. The term “treatment”, as used herein, refers to the act of treating, as “treating” is defined immediately above.

“Optional” or “optionally” means that the subsequently described element, component or circumstance may or may not occur, so that the description includes instances where the element, component, or circumstance occurs and instances where it does not.

The terms “subject,” “individual” or “patient” are used interchangeably herein and refer to a vertebrate, such as a mammal. Mammals include, but are not limited to, humans.

As used herein, the term “composition” is intended to encompass a product comprising the specified ingredients in the specified amounts, as well as any product which results, directly or indirectly, from combinations of the specified ingredients in the specified amounts.

The terms “drug,” “active agent,” “therapeutic agent,” and/or “pharmacologically active agent” are used interchangeably herein to refer to any chemical compound, complex, or composition that is suitable for oral administration and that has a beneficial biological effect, such as a therapeutic effect in the treatment or prevention of a disease or abnormal physiological condition. The terms also encompass pharmaceutically acceptable, pharmacologically active derivatives of those active agents specifically mentioned herein, including, but not limited to, salts, esters, amides, prodrugs, active metabolites, analogs, conjugates, and the like. When the terms “active agent,” “pharmacologically active agent,” and “drug” are used, then, or when a particular active agent is specifically identified, it is to be understood that applicants intend to include the active agent per se as well as pharmaceutically acceptable, pharmacologically active salts, esters, amides, prodrugs, metabolites, analogs, conjugates, etc.

The term “dosage form” denotes any form of a pharmaceutical composition that contains an amount of active agent sufficient to achieve a therapeutic effect with a single administration. When the formulation is a tablet or capsule, the dosage form can be one or more such tablets or capsules.

The term “dosage unit” refers to a single unit of the dosage form that is to be administered to the patient. The dosage unit will be typically formulated to include an amount of drug sufficient to achieve a therapeutic effect with a single administration of the dosage unit although where the size of the dosage form is at issue, more than one dosage unit may be necessary to achieve the desired therapeutic effect. For example, a single dosage unit of a drug is typically, one tablet, one capsule, or one tablespoon of liquid. More than one dosage unit may be necessary to administer sufficient drug to achieve a therapeutic effect where the amount of drug causes physical constraints on the size of the dosage form.

The terms “effective amount” or a “therapeutically effective amount” refer to the amount of drug or pharmacologically active agent that is effective to achieve a desired therapeutic result. The amount of an agent that is “effective” may vary from individual to individual, depending on the age, weight, general condition, and other factors of the individual. An appropriate “effective” amount in any individual may be determined by one of ordinary skill in the art using routine experimentation. An “effective amount” of an agent can refer to an amount that is either therapeutically effective or prophylactically effective or both.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative, refers lo a derivative having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA.

The term “biocompatible” is used interchangeably with the term “pharmaceutically acceptable.”

A drug “release rate,” as used herein, refers to the quantity of drug released from a dosage form or pharmaceutical composition per unit time, e.g., milligrams of drug released per hour (mg/hr), Drug release rates for drug dosage forms are typically measured as an in vitro rate of dissolution, i.e., a quantity of drug released from the dosage form or pharmaceutical composition per unit time measured under appropriate conditions and in a suitable fluid.

The term “controlled release” is intended to refer to any drug-containing formulation in which release of the drug is not immediate, i.e., with a “controlled release” formulation, oral administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995).

The terms “sustained release,” and “extended release” are used interchangeably herein to refer to a dosage form that provides for release of a drug over an extended period of time. With extended release dosage forms, the rate of release of the drug from the dosage form is reduced in order to maintain therapeutic activity of the drug for a longer period of time or to reduce any toxic effects associated with a particular dosing of the drug. Extended release dosage forms have the advantage of providing patients with a dosing regimen that allows for less frequent dosing, thus enhancing compliance. Extended release dosage forms can also reduce peak-related side effects associated with some drugs and can maintain therapeutic concentrations throughout the dosing period thus avoiding periods of insufficient therapeutic plasma concentrations between doses.

“Delayed release” dosage forms are a category of modified release dosage forms in which the release of the drug is delayed after oral administration for a finite period of time after which release of the drug is unhindered. Delayed release dosage forms are frequently used to protect an acid-labile drug from the low pH of the stomach or where appropriate to target the GI tract for local effect while minimizing systemic exposure. Enteric coating is frequently used to manufacture delayed release dosage forms.

The term “modified release” encompasses all nonimmediate release drug products, The manufacture of delayed, extended, and modified release dosage forms are known to ordinary skill in the art and include the formulation of the dosage forms with excipients or combinations of excipients necessary to produce the desired active agent release profile for the dosage form.

The “gastric retentive” oral dosage forms described herein are a type of extended release dosage form, Gastric retentive dosage forms are beneficial for the delivery of drugs with reduced absorption in the lower GI tract or for local treatment of diseases of the stomach or upper GI tract. For example, in certain embodiments of gastric retentive oral dosage forms, the dosage form swells in the gastric cavity and is retained in the gastric cavity of a patient in the fed med so that the drug may be released for heightened therapeutic effect (Hou et al., Crit. Rev. Ther, Drug Carrier Syst, 2003, 20(6):459-497). In other embodiments, enlarging dosage forms, floating dosage forms, including gas generating dosage forms, and bioadhesive dosage forms have been described as gastric retentive dosage forms.

The term “AUC” (Le., “area under the curve,” “area under the concentration curve,” or “area under the concentration-time curve”) is a pharmacokinetic term used to refer a method of measurement of bioavailability or extent of absorption of a drug based on a plot of an individual or pool of individual's blood plasma concentrations sampled at frequent intervals; the AUC is directly proportional to the total amount of unaltered drug in the patient's blood plasma. For example, a linear curve for a plot of the AUC versus dose (i.e., straight ascending line) indicates that the drug is being released slowly into the blood stream and is providing a steady amount of drug to the patient; if the AUC versus dose is a linear relationship this generally represents optimal delivery of the drug into the patient's blood stream. By contrast, a non-linear AUC versus dose curve indicates rapid release of drug such that some of the drug is not absorbed, or the drug is metabolized before entering the blood stream. The total area under the curve from time zero to time infinity is referred to as AUCinf. Partial AUCs can be calculated for specific intervals of time, i.e., AUC(0-6 h) means the area under the curve from time zero to time six hours after dosing.

The term “Cmax” (Le., “maximum concentration”) is a pharmacokinetic term used to indicate the peak concentration of a particular drug in the blood plasma of a patient. The term “Cmin” (i.e., “minimum concentration”) is a pharmacokinetic term used to indicate the minimum concentration of a particular drug in the blood plasma of a patient.

The term “Tmax” (i.e., “time of maximum concentration” or “time of Cmax”) is a pharmacokinetic term used to indicate the time at which the Cmax is observed during the time course of a drug administration.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0. The polymeric carriers herein are hydrophilic, and thus compatible with aqueous fluids such as those present in the human body.

The terms “peptide” and “polypeptide” refer to a polymer of two or more amino acids joined to each other by peptide bonds or modified peptide bonds, i.e., peptide isosteres. Peptides are distinguished from polypeptides on the basis of size, and typically contain fewer monomer units than polypeptides. The size boundaries which distinguish peptides and polypeptides are arbitrary. As used herein, the term peptide encompasses amino acid polymers of any length from about two to about two hundred amino acids. The terms apply to amino acid polymers containing naturally occurring amino acids as well as amino acid polymers in which one or more amino acid residues is a non-naturally occurring amino acid or a chemical analog of a naturally occurring amino acid. An amino acid polymer may contain one or more amino acid residues that has been modified by one or more natural processes, such as post-translational processing, and/or one or more amino acid residues that has been modified by one or more chemical modification techniques known in the art. Certain exemplary modifications include, but are not limited to, acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demeihylation, formation of covalent cross-links, formation of cystine, formation of pyroglutamate, formylation, gamma-carboxylation, glycosylation, GPI anchor formation, hydroxylation, iodination, methylation, myristoylation, oxidation, octanoylation, proteolytic processing, phosphorylation, prenylation, racemization, selenoylation, sulfation, transfer-RNA mediated addition of amino acids to proteins such as arginylation, and ubiquitination. The terms also applies to amino acid polymers in which one or more amino acid residue is an artificial chemical mimetic of a corresponding naturally occurring amino acid.

The term “amino acid” refers to naturally occurring and non-natural amino acids, as well as amino acid analogs and amino acid mimetics that function in a manner similar to the naturally occurring amino acids.

Naturally occurring amino acids are those encoded by the genetic code, as well as those amino acids that are later modified, e.g., hydroxyproline, ,gamma.-carboxyglutamate, and O-phosphoserine.

Non-natural amino acid, as used herein, refers to any amino acid, modified amino acid, and/or amino acid analog that is not one of the naturally occurring amino acids.

Amino acid analog refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “peptide analog” refers to compounds that have the same basic chemical structure as a naturally occurring peptide, i.e. amino acids linked via amide bonds, where one or more amino acids is a non-natural amino acid.

The term “peptide mimetic” refers to compounds where the structure of the compound is different from the general chemical structure of a peptide, but the compound functions in a manner similar to a peptide. It is understood that when the term “peptide” is used herein, it is intended to encompass peptides, peptide analogs, and peptide mimetics.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “variant” of a reference peptide refers to a peptide having one or more amino acid substitutions, deletions, or insertions relative to the reference peptide. In certain embodiments, a variant of a reference peptide has an altered post-translational modification site (i.e., a glycosylation site).

A “derivative” of a reference peptide refers to: a peptide: (1) having one or more modifications of one or more amino acid residues of the reference peptide; and/or (2) in which one or more peptidyl linkages has been replaced with one or more non-peptidyl linkages; and/or (3) in which the N-terminus and/or the C-terminus has been modified.

“Conservatively modified variants,” as used herein refers to peptides, polypeptides, or proteins in which individual substitutions, deletions or additions alter, add or delete a single amino acid or a small percentage of amino acids in the peptide, polypeptide or protein sequence, where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (5), Threonine (T); and 8) Cysteine (C), Methionine (M) (see, e.g., Creighton, Proteins (1984)).

The term “polymer” as used herein refers to a molecule containing a plurality of covalently attached monomer units, and includes branched, dendrimeric and star polymers as well as linear polymers. The term also includes both homopolymers and copolymers, e.g., random copolymers, block copolymers and graft copolymers, as well as uncrosslinked polymers and slightly to moderately to substantially crosslinked polymers, as well as two or more interpenetrating cross-linked networks.

The term “swellable polymer,” as used herein, refers to a polymer that will imbibe a fluid, such as water, and become enlarged or engorged. A polymer is swellable due, at least in part, to a structural feature of the polymer. It is understood that a given polymer may or may not swell when present in a defined drug formulation. Accordingly, the term “swellable polymer” defines a structural feature of a polymer which is dependent upon the composition in which the polymer is formulated. Whether or not a swellable polymer when incorporated into a dosage form or matrix containing other components swells in the presence of fluid will depend upon a variety of factors, including the specific type of polymer and the percentage of that polymer in a particular formulation. For example, the term “polyethylene oxide” or “PEO” refers to a polyethylene oxide polymer that has a wide range of molecular weights. PEO is a linear polymer of unsubstituted ethylene oxide and has a wide range of viscosity-average molecular weights. Examples of commercially available PEOs and their approximate molecular weights are: POLYOX® NF, grade WSR coagulant, approximate molecular weight 5 million daltons (Da), POLYOX® grade WSR 301, approximate molecular weight 4 million Da, POLYOX® grade WSR 303, approximate molecular weight 7 million Da, POLYOX® grade WSR N-60K, approximate molecular weight 2 million Da, and POLYOX® grade N-80K, approximate molecular weight 200,000 Da. An oral dosage form which comprises a swellable polymer as used herein intends that the polymer when incorporated into the dosage form will swell upon imbibition of water or fluid from gastric fluid.

The term “fed mode,” as used herein, refers to a state which is typically induced in a patient by the presence of food in the stomach, the food giving rise to two signals, one that is said to stem from stomach distension and the other a chemical signal based on food in the stomach. It has been determined that once the fed mode has been induced, larger particles are retained in the stomach for a longer period of time than smaller particles. Thus, the fed mode is typically induced in a patient by the presence of food in the stomach.

Administration of a dosage form “with a meal,” as used herein, refers to administration before, during or after a meal, and more particularly refers to administration of a dosage form about 1 minute (min), 2 min, 3 min, 4 min, 5 min, 10 min, 15 minutes before commencement of a meal, during the meal, or about 1 min, 2 min, 3 min, 4 min, 5 min, 10 min, 15 min after completion of a meal.

### Methods of Treatment

Also contemplated are methods for treating GI disorders by orally administering to a subject in need thereof, a therapeutically effective amount of a composition comprising an active agent for the treatment of a GI disorder. As used herein the words “treat,” “treating”, “treatment”, or “therapeutic treatment” refer to using the compositions either prophylactically to prevent or reduce incidence of the development of symptoms, or therapeutically to ameliorate an existing condition characterized by symptoms of GI motility dysfunction, such as gastroparesis.

Dosage forms comprising an active agent, as described in more detail below, are provided to subjects suffering from or diagnosed with a GI motility disorder.

In one aspect, a method for treating a subject suffering from a GI motility disorder comprising orally administering an ER active agent dosage form is provided.

In one embodiment, an ER dosage form comprising an active agent is administered to a subject suffering from or diagnosed with a GI motility disorder.

In another embodiment, a gastric retained dosage form comprising an active agent and at least one swellable polymer is administered to a subject suffering from or diagnosed with a GI motility disorder.

In yet another embodiment, an ER dosage form comprising an active agent is administered with a second active agent to a subject suffering from or diagnosed with a GI motility disorder.

In still another embodiment, an ER dosage form comprising an active agent and a second active agent is administered to a subject suffering from or diagnosed with a GI motility disorder.

The active agent may be used for treating a subject suffering from or diagnosed with a GI motility disorder. All types of GI motility disorders are envisioned including, but not limited to, irritable bowel syndrome, constipation, gastroparesis, GERD, emesis, ileus, gastritis, and dyspepsia.

In determining what constitutes an effective amount or a therapeutically effective amount of active agent, for treating one or more of the disorders or conditions referred to above, a number of factors will generally be considered by the medical practitioner or veterinarian in view of the experience of the medical practitioner or veterinarian, published clinical studies, the subject's age, sex, weight and general condition, as well as the type and extent of the disorder or condition being treated, and the use of other medications, if any, by the subject. As such, the administered dose may fall within the ranges or concentrations recited above, or may vary outside, i.e., either below or above, those ranges depending upon the requirements of the individual subject, the severity of the condition being treated, and the particular therapeutic formulation being employed. Determination of a proper dose for a particular situation is within the skill of the medical or veterinary arts. Generally, treatment may be initiated using smaller dosages of active agent that are less than optimum for a particular subject. Thereafter, the dosage can be increased by small increments until the optimum effect under the circumstance is reached. For convenience, the total daily dosage may be divided and administered in portions during the day, if desired.

In assessing efficacy of the active agent in the treatment of GI motility disorders, a variety of measures typically used by physicians for evaluating and grading the symptoms associated with the disorders can be used. Changes in the assessments over time are compared for various treatments to determine efficacy. For example, changes in gastric motility can be evaluated by measuring gastric emptying rate using a gastric emptying breath test, gastric half emptying time (GET_{1/2}), bowel movement parameters (time to first bowel movement after first dose, bowel movement count, stool consistency), changes from baseline over time in daily symptom scores from a self administered daily symptom diary, and rate of adequate relief using physician or investigator global symptom assessments.

It is understood that treatment of the above disorders with a therapeutically effective amount of active agent will result, for example, in a significant decrease in the symptoms of the disorders. For example, providing a therapeutically effective amount of an active agent for treating gastroparesis will result in an increase in the gastric emptying rate.

Therapeutically effective amounts of the active agent administered as described herein will generally be from about 0.01 mg/kg to about 75 mg/kg of subject body weight. Typical doses will be from about 5 mg/day to about 5000 mg/day for an adult subject of normal weight. In a clinical setting, regulatory agencies such as, for example, the Food and Drug Administration (“FDA”) in the U.S. may require a particular therapeutically effective amount.

### Dosage Forms

### I. Extended Release Dosage Forms

The transit time through the GI tract often limits the amount of drug available for absorption at its most efficient site of absorption, or for local activity at one segment of the GI tract. This is particularly true when the absorption/activity site is high in the GI tract. Extended release dosage forms may provide a benefit over immediate release dosage forms for active agents that are absorbed or active locally in the upper GI tract, especially if they are retained in the upper GI tract for an extended period of time. The dosage forms described herein are effective for the continuous, controlled administration of drugs which act either locally within the GI tract or systemically by absorption into the circulation via the GI mucosa. The dosage forms are also useful for the delivery of drugs that have been granulated, encapsulated, included in a particle, or coated with enteric coating material, so that they pass from the acidic environment of the stomach before they dissolve, thus protecting the drugs from acid and enzymes in the stomach and providing continuous delivery of the drugs to the upper part of the small intestine in a controlled manner.

In addition, modified release dosage forms that are administered once- or twice-daily offer advantages over their immediate release counterparts because they reduce the magnitude of peaks and troughs of drug plasma concentration, provide longer dosing intervals, sustained therapeutic effect, and increased patient compliance. These modified release formulations may be referred to as controlled release (CR), sustained release (SR) and/or extended release (ER), etc. Methods for preparing MR formulations are well known in the pharmaceutical arts. In performing the method described herein, any type of modified release mechanism may be used for the active agent dosage form, including, but not limited to, osmotic, coating, polymer matrix, multiparticulate, erosional, diffusional, gastric retention via floating, swelling, expansion, and/or gas-generation, and rate-controlling membrane mechanisms. Such mechanisms are well known to those skilled in the art of ER oral dosage forms. The ER compositions disclosed herein may comprise an immediate release portion.

Solid oral dosage forms useful for administering active agent include capsules, caplets, tablets, and powders. Fillers, binders, lubricants, disintegrants, and other additives may also be included in the dosage form, such as are well known to those of skill in the art. The dosage form may be a single layer, bilayer, or multilayer tablet or it may be a capsule. The formulations may assume the form of particles, tablets, or particles retained in capsules. In certain embodiments a formulation comprises particles consolidated into a packed mass for ingestion, even though the packed mass will separate into individual particles after ingestion. Conventional methods can be used for consolidating the particles in this manner. For example, the particles can be placed in gelatin capsules known in the art as “hard-filled” capsules and “soft-elastic” capsules. The compositions of these capsules and procedures for filling them are known among those skilled in drug formulations and manufacture. The encapsulating material is preferably highly soluble so that the particles are freed and rapidly dispersed in the stomach after the capsule is ingested.

In certain embodiments, the ER dosage form contains an additional amount of the drug applied as a quickly dissolving coating on the outside of the particle or tablet. This may be the same drug as the drug in the ER portion or a different drug.

In a first aspect, the ER active agent dosage form is a capsule. In certain embodiments, the dosage form is a capsule comprising an ER portion. In other embodiments, the dosage form is a capsule comprising an ER portion and an IR portion.

A film coating may also be included on the outer surface of the dosage form for reasons other than to provide immediate release of drug. The coating may thus serve an aesthetic function or a protective function, or it may make the dosage form easier to swallow or mask the taste of the drug.

In another aspect, swellable polymer matrix ER active agent dosage forms are formulated. Gastric retained dosage forms that can form the basis for the sustained release of a drug have been previously described, for example, in Gusler et al. (U.S. Pat. No. 6,723,340), Berner et al, (U.S. Pat. No. 6,488,962), Shell et al., (U.S. Pat. No. 6,340,475) and Shell et al. (U.S. Pat. No. 6,635,280), all of which are herein incorporated by reference. These formulations make use of one or more hydrophilic polymers which swell upon intake of water from gastric fluid. Gastric retentive dosage forms are generally applicable to drugs where the bioavailability improves when administered with a meal. Thus, when administered in the fed mode, when the diameter of the pyloric sphincter is contracted and reduced, the dosage form will swell, preferably unrestrained dimensionally, to a size to be retained in the stomach for a minimum of four hours or more. These formulations may be designed to produce desired release and delivery profiles for both highly soluble and poorly soluble drugs.

Other types of gastric retentive dosage forms include floating, bioadhesive, and expanding (non-swelling) dosage forms. Floating dosage forms rely on remaining physically distant from the pylorus to avoid premature emptying from the stomach. Floating is most often accomplished by inclusion of a gas-generating agent in the dosage form. Another approach to retention in the upper GI tract is the use of bioadhesive dosage forms which rely on the presence of bioadhesive additives for physical attachment to the mucus or mucosa lining in the upper GI tract by sticking to the mucus and/or mucosa lining. Dosage forms which rapidly expand include those which expand by unfolding (“accordion”), or by inclusion of a gas-generating agent.

Gastric retentive dosage forms formulated specifically to provide extended release of gabapentin, an antiepileptic agent, from the stomach into the upper gastrointestinal tract are disclosed in Berner et al. (U.S. Pat. Nos. 7,438,927, and 7,731,989), herein incorporated by reference. These dosage forms result in prolonged exposure and lower but steady release rate of the active agent to the small intestine to optimize uptake and enhance bioavailability, and are administered with a meal.

In another aspect, a gastric retained extended release (ER) dosage form comprising a dose of active agent dispersed in a polymer matrix comprising at least one hydrophilic polymer is provided. Upon administration, the polymer matrix swells upon imbibition of water to a size sufficient such that the dosage form is retained in the stomach of a subject in a fed mode and the dose of active agent is released over an extended period of time.

In still another aspect, a ER dosage form is provided which releases an active agent primarily via erosion of the dosage form, as disclosed in Berner et al. (U.S. Pat. No. 7,976,870), herein incorporated by reference. This dosage form comprises a matrix of a hydrophilic swellable polymer with an active agent incorporated therein. The polymer is one that both swells in the presence of water and gradually erodes over a period of hours with the drug release rate primarily controlled by the polymer erosion rate.

In yet another aspect, a dosage form is formulated to have a dual-matrix configuration (“shell-and-core”) as described in U.S. Pat. No. 7,736,667 and U.S. Pat. No. 8,043,630 (herein incorporated by reference). One matrix forms a core of polymeric material in which drug is dispersed and the other matrix forms a casing that surrounds and fully encases the core, the casing being of polymeric material or membrane that swells upon imbibition of water (and hence gastric fluid) to a size large enough to promote retention in the stomach during the fed mode, the shell and core being configured such that the drug contained in the core is released from the dosage form by diffusion through the shell. The core polymer may also be a swelling polymer, and if so, compatible polymers will be selected that will swell together without disrupting the integrity of the shell. The core and shell polymers may be the same or different, and if the same, they may vary in molecular weight, crosslinking density, copolymer ratio, or any other parameter that affects the swelling rate, so long as any swelling occurring in the core does not cause substantial splitting of the shell. The shell is of sufficient thickness and strength that it is not disrupted by the swelling and remains intact during substantially the entire period of drug release. The shell may or may not contain drug.

An alternative oral dosage formulation may be the preparation of a tablet which has an immediate release (IR) core containing a drug, completely surrounded or encased by an extended release (ER) shell, wherein the ER shell also contains the drug and swells upon imbibition of water, The ER shell can be designed to swell rapidly enough to a size sufficient for gastric retention in the stomach of a subject in the fed mode. This alternative “pulsatile release” dosage form may be useful to ensure that levels of the drug released from the dosage form are maintained at therapeutically effective levels. For example, if towards the end of the time period of sustained or extended release of the drug from the ER shell, the drug that has reached the blood of the subject has metabolized or been excreted to a level below that which has optimal therapeutic effect, the subsequent burst of the drug dose from the IR core will bring the drug to a more therapeutic level in the blood.

One having ordinary skill in the art would understand that this dosage form may further comprise an IR coat applied to the surface of the ER shell. This IR coat could contain the same drug or a different drug, and the IR coat would dissolve to immediately release the drug into the stomach after oral administration of the tablet.

In another aspect, the ER dosage form is an osmotic dosage form such as an elementary osmotic dosage form or a push-pull osmotic pump. For example, U.S. Pat. Nos. 3,845,770 and 3,916,899 issued to Theeuwes and Higuchi pertain to an osmotic dosage form for delivering various drugs to a patient. The dosage forms disclosed in these patents consist of a wall that surrounds a compartment comprising a drug with an exit in the wall for delivering the drug to a patient. Osmotic dosage forms comprising a drug compartment and a pharmaceutically acceptable polymer hydrogel (maltodextrin, polyalkylene oxide, polyethylene oxide, carboxyalkylcellulose), contained within a bilayer interior wall and exterior wall and having a passageway, where the polymer exhibits an osmotic pressure gradient across the bilayer interior wall and exterior wall thereby imbibing fluid into the drug compartment to form a solution or a suspension comprising the drug that is hydrodynamically and osmotically delivered through a passageway from the dosage form are disclosed by Edgren et al. (U.S. Pat. No. 6,245,357). In certain embodiments, the dosage form further comprises a push displacement layer which expands to expel the drug from the dosage form.

Tablets are one form of the ER active agent dosage forms contemplated. In certain embodiments, the dosage form is a pharmaceutical tablet for the extended release of the active agent. In one embodiment, the tablet comprises an ER portion. In another embodiment, the dosage form is a single layer tablet In another embodiment, the dosage form comprises a coat. In yet another embodiment, the coat comprises one or more active agents. In still another embodiment, the coat comprises the active agent.

In another aspect of the disclosure, the tablet comprises an ER portion and an IR portion. In one embodiment, the tablet is a bilayer tablet, comprising an ER portion and an IR portion. In certain embodiments, the dosage form is a bilayer tablet, wherein one or more layers comprise active agent. In one embodiment, one layer or more layers comprise an additional active agent.

In yet another aspect, the dosage form is a bilayer or multilayer tablet. In one embodiment, the dosage form comprises a layer which is a floating layer, with or without an active agent. In yet another embodiment, the dosage form comprises a layer which is a swelling layer, with or without an active agent. In still another embodiment, the dosage form comprises a layer which is an IR layer.

In still another aspect, the dosage form comprises an immediate release matrix containing the active agent, surrounded by a rate-limiting membrane. In one embodiment, the rate-limiting membrane functions as a semi-permeable membrane when immersed in fluid.

In one embodiment, at least 50%, 60%, 70%, 80%, or 85% of the active agent is released from the tablet over a time period of about 2 h to 12 h (hours), 3 h to 6 h, 2 h to 4 h, 3 h to 7 h, 5 h to 8 h, 2 h to 8 h, or 2 h to 10 h. In another embodiment, at least about 80%, 85%, 90%, 95%, 96%, 97%, 98%, or 99% of the active agent is delivered to the GI tract of the subject over a time period of at least 1 h, 2 h, 3 h, 4 h, 5 h, 6 h, 7 h, 8 h, 9h, 10 h, 11 h, or 12 h.

In one aspect, the ER dosage form is a tablet. In one embodiment, the total tablet weight is about 500 mg or about 1000 mg (milligrams). In another embodiment, the total tablet weight is about 1200 mg. In yet another embodiment, the total tablet weight is about 500 mg to about 1500 mg, 750 mg to 1500 mg, 800 mg to 1300 mg, 900 mg to 1250 mg, or about 800 to 1200 mg.

In one embodiment, the tablet comprises about 380 mg, 400 mg, 420 mg, 440 mg, 460 mg, 480 mg, 500 mg, 520 mg, 540 mg, 560 mg, 580 mg, 600 mg, or 620 mg of one or more hydrophilic polymers. In another embodiment, the tablet comprises about 15 wt % to 50 wt %, 15 wt % to 40 wt %, 20 wt % to 30 wt %, 20 wt % to 40 wt %, 25 wt % to 45 wt %, or 30 wt % to 50 wt % of a hydrophilic polymer. In yet another embodiment, the tablet comprises about 15 wt %, 18 wt %, 20 wt %, 25 wt %, 28 wt %, 30 wt %, 32 wt %, 33 wt %, 35 wt %, 37 wt %, 40 wt % or 90 wt % of a hydrophilic polymer.

In one embodiment, the tablet comprises one or more hydrophilic polymers, each having an average molecular weight ranging from about 200,000 Da (Daltons) to about 10,000,000 Da, about 900,000 Da to about 5,000,000 Da, about 2,000,000 Da to about 5,000,000 Da, from about 4,000,000 Da to about 5,000,000 Da, from about 5,000,000 Da to about 7,000,000 Da, from about 2,000,000 Da to about 4,000,000 Da, from about 900,000 Da to about 5,000,000 Da, or from about 900,000 Da to about 4,000,000 Da. In another embodiment, the tablet comprises a hydrophilic polymer having an average molecular weight of about 200,000 Da, 600,000 Da, 900,000 Da, 1,000,000 Da, 2,000,000 Da, 4,000,000 Da, 5,000,000 Da, 7,000,000 Da, 10,000,000 Da or 12,000,000 Da.

In one embodiment, the ER layer comprises a hydrophilic polymer having an average viscosity ranging from about 4,000 cPs (centipoise) to about 200,000 cPs, from about 50.000 cPs to about 200,000 cPs, or from about 80,000 cPs to about 120,000 cPs as measured as a 2% weight per volume in water at 20° C.

In one embodiment, the one or more hydrophilic polymers in the tablet is a polyalkylene oxide. In another embodiment, the hydrophilic polymer is poly(ethylene oxide), In yet another embodiment, the at least one hydrophilic polymer in the tablet is a cellulose. In yet another embodiment, the cellulose is hydroxypropylmethylcellulose.

In one embodiment, the tablet comprises two hydrophilic polymers in a ratio of 1:1, 1.2:1, 1.3:1, 1.4:1, 1.5:1, 1.6:1, 1.8:1, or 2.0:1.

### II. Excipients

The ER dosage form may comprise additional excipients. Suitable excipients include binders, fillers, disintegrants, lubricants, buffering agents, antioxidants, chelating agents, solubilizing agents and color agents.

Binders are used to impart cohesive qualities to a tablet, and thus ensure that the tablet or tablet layer remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone (including povidone and copovidone), cellulosic polymers (including hydroxypropyl cellulose, hydroxypropyl methylcellulose, methyl cellulose, microcrystalline cellulose, ethyl cellulose, hydroxyethylcellulose, and the like), and Veegum.

In one embodiment, the tablet comprises about 20 mg, 25 mg, 30 mg, 35 mg, 40 mg, 45 mg, or 50 mg of one or more binders. In another embodiment, the tablet comprises about 1 wt % to 5 wt %, 1 wt % to 4 wt %, 1.5 wt % to 3 wt %, or 2 wt % to 3 wt %. hi another embodiment, the tablet comprises about 1 wt %, 2 wt %, 2.5 wt %, 3 wt %, 3.5 wt %, 4 wt %, 4.5 wt %, 5 wt %, or 6 wt % binder. In yet another embodiment, the tablet comprises about 1 wt % to about 6 wt % or about 2 wt % to about 5 wt % of a binder.

In one embodiment, the tablet comprises a binder which is polyvinylpyrrolidone, polyvinylalcohol, ethyl cellulose, hydroxypropylcellulose, hydroxypropylmethylcellulose, hydroxyethylcellulose or polyethylene glycol. In yet another embodiment, the polyvinylpyrrolidone is povidone or copovidone. In yet another embodiment, the tablet comprises a combination of more than one binder.

Lubricants are used to facilitate tablet manufacture, promoting powder flow and preventing particle capping (i.e., particle breakage) when pressure is relieved. Useful lubricants are magnesium stearate (in a concentration of from 0.25 wt % to 3 wt %, preferably 0,2 wt % to 1.0 wt %, more preferably about 0.3 wt %), calcium stearate, stearic acid, and hydrogenated vegetable oil (preferably comprised of hydrogenated and refined triglycerides of stearic and palmitic acids at about 1 wt % to 5 wt %, most preferably less than about 2 wt %).

In one embodiment, the tablet comprises about 8 mg, 9 mg, 10 mg, 11 mg, 12 mg, 13 mg, 14 mg, 15 mg of one or more lubricants. In another embodiment, the tablet comprises about 0.5 wt % to 2.5 wt %, 1 wt % to 2 wt % or 0.5 wt % to 2 wt % of a lubricant. hi yet another embodiment, the tablet comprises about 0.5 wt %, 1.0 wt %, 1.5 wt %, 2.0 wt %, or 2.5 wt % of a lubricant.

In one embodiment, the tablet comprises a lubricant which is magnesium stearate, calcium stearate, sodium stearyl fumarate, stearic acid, stearyl behenate, glyceryl behenate, or polyethylene glycol.

In one embodiment, the dosage form may comprise a buffering agent. Buffers are used in oral dosage forms to alter the pH in the dosage form and/or in the local environment of the stomach, which can modify the stability and/or the solubility of a drug. The buffering agent will be present in the dosage form in an amount within the range of from about 1 to about 30% by weight and preferably from about 2 to about 15% by weight of the composition. Examples of buffering agents which may be used in the dosage forms include, but are not limited to, calcium carbonate, magnesium carbonate, sodium acetate, sodium bicarbonate, sodium borate, sodium carbonate, sodium citrate, sodium tartrate, sodium fumarate, sodium malate, sodium succinate, magnesium oxide, aluminum hydroxide, dihydroxyaluminum sodium carbonate, an alkaline earth metal hydroxide such as calcium hydroxide or magnesium hydroxide, and ionizable amino acids. Examples of amino acids useful as buffering agents include glutamic acid, glutamine, glycine, aspartic acid, alanine and arginine, as well as salts thereof. Mixtures of one or more of the above-mentioned buffer agents may also be used.

In yet another embodiment, the dosage form may comprise at least one antioxidant. The dosage form may contain in the IR layer, the ER layer, or both layers, an anti-oxidant for increased stability of the active ingredient and/or as the dosage form as a whole. Suitable antioxidants include, without limit, ascorbic acid, citric acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfite, sorbic acid, potassium ascorbate, vitamin E, 4-chloro-2,6-ditertiarybutylphenol, alphatocopherol, propylgallate, and sulfur-containing antioxidant compounds such as cysteine, methionine, taurine, glutathione, lipoic acid, mercaptopropionylglycine, and N-acetylcysteine or any combination of the above. The amount of antioxidant present in a tablet may range from about 0.01% to about 4.0% (w/w), or from about 0.02% to about 0.10% (w/w). In various embodiments, the amount of antioxidant present in the tablet may be about 0.01%, 0.02%, 0.03%, 0.04%, 0.05%, 0.06%, 0.07%, 0.08%, 0.09%, 0.10%, 0.12%, 0.14%, 0.16%, 0.18%. 0.20%, 0.25%, 0.50%, 0.75%, 1.00%, 1.50%, or 2.00% (w/w) of the total weight of the tablet.

In one embodiment, the dosage form comprises a chelating agent. Chelating agents tend to form complexes with trace amounts of heavy metal ions inactivating their catalytic activity in the oxidation of medicaments. Ethylenediamine tetracetic acid (EDTA), its derivatives and their salts, dihydroxy ethyl glycine, citric acid and tartaric acid are most commonly used chelators.

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinylpyrrolidone). In one embodiment, the dosage form comprises a disintegrant which is cellulose or a derivative of cellulose such as microcrystalline cellulose or carboxymethyl cellulose, crospovidone, crosslinked starch such as sodium starch glycolate, alginic acid or soy polysaccharides.

Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions.

In one embodiment, the tablet comprises one or more additional excipients which are diluents, solubilizing agents, coloring agents, flavoring agents, stabilizers, and/or glidants.

### III. Enteric Coatings

As described above, the oral ER dosage forms may comprise an active agent dispersed within a swellable hydrophilic polymer matrix. Upon administration, the dosage form imbibes water and swells to a size sufficient for retention of the dosage form in the stomach in a fed mode. The active agent is then released over a time period of about 2 hours to 4 hours, about 3 hours to about 12 hours, about 1 hour, about 2 hours, about 3 hours, about 4 hours, about 5 hours, about 6 hours, about 7 hours or about 8 hours. The active agent may be either a plurality of particles comprised of active agent (active agent particles) or a plurality of micropellets comprising active agent. The active agent particles and the micropellets are each of a size such that they pass through the pylorus to the small intestine essentially immediately upon release from the dosage form.

In some embodiments, the active agent particles and micropellets comprise an enteric coating. Enteric coatings will remain intact in the stomach but will rapidly dissolve once they arrive at the small intestine, thereafter releasing the drug at sites downstream in the intestine (e.g., the ileum and colon). Enteric coatings are well known in the art and are discussed at, for example, Remington's Pharmaceutical Sciences, Mack Publishing Co., Easton, Pa.; and Polymers for Controlled Drug Delivery, Chapter 3, CRC Press, 1991. Methods for applying enteric coatings to pharmaceutical compositions are well known in the art, and include for example, U.S. Parent Publication No. 2006/0045822.

Enteric coatings have long been used to inhibit release of drug from tablets and pellets in order to protect the drug from degradation in the stomach. This approach is particularly useful for the delivery of peptides which are subject to enzymatic degradation as well as acid-catalyzed hydrolysis in the stomach and has even been employed to target delivery of peptides or proteins to the colon. The enteric coatings are resistant to stomach acid for required periods of time depending on the composition and/or thickness thereof, before they begin to disintegrate and allow for slow release of drug in the stomach and/or upper intestines. Some examples of the first enteric coatings used are beeswax and glyceryl monostearate; beeswax, shellac, cellulose; and cellulose acetate phthalate, and cetyl alcohol, mastic and shellac as well as shellac and stearic acid (U.S. Pat. No. 2,809,918); polyvinylacetate and ethyl cellulose (U.S. Pat. No, 3,835,221); and neutral copolymer of polymethacrylic acid esters (Eudragit L30D). (F. W. Goodhart et al, Pharm. Tech., p. 64-71, April, 1984); copolymers of methacrylic acid and methacrylic acid methyl ester (Eudragits), or a neutral copolymer of polymethacrylic acid esters containing metallic stearates (Mehta et al U.S. Pat. Nos. 4,728,512 and 4,794,001), and hypromellose phthalate. Most available enteric coating polymers begin to become soluble at pH 5.5 and above, with maximum solubility rates at pH values greater than 6.5.

As a means of optimizing the release rate of an active agent, it may be advantageous to liberate the active agent from the enteric bead as fast as possible after passing through the pylorus into the proximal small intestine. As a means of accelerating the rate of dissolution of the enteric coat, a half-thickness, double coat of enteric material (for instance, Eudragit L30 D-55) may be applied, wherein the inner enteric coat is buffered up to pH 6.0 in the presence of 10% citric acid, followed by a final layer of standard Eudragit L 30 D-55. Applying two layers of enteric coat, each half the thickness of a typical enteric coat, the team of Liu and Basit were able to demonstrate accelerated enteric coating dissolution compared to a similar coating system applied, unbuffered, as a single layer (Liu, F. and Basil, A. Journal of Controlled Release. 147 (2010) 242-245).

In general, known enteric coatings require addition of a plasticizer to avoid cracking, in particular, during the stress caused by compression in the tableting process. Accordingly, it is important that the enteric coating of the active agent-containing micropellets remains intact during the process of manufacturing the ER dosage form and consequently, most formulations include a plasticizing excipient, most commonly triethyl citrate.

The processes described herein for manufacturing an ER dosage form comprising micropellets or beads dispersed within a polymeric matrix may involve at least dry or wet blending, or other such tableting processes. Exposure of the micropellets would be expected by an ordinary artisan to be subject to breakage or other more subtle structural compromise. Any such breakage would expose the active agent contained within the micropellets to the highly acidic environment of the stomach, leading to inactivation and/or degradation of the active agent before it reaches the small intestine.

In some embodiments, the dosage forms described herein comprise a plurality or enteric-coated micropellets, beads, or particles in which the enteric coating for each of the micropellets or particles remains substantially intact. When the enteric coating for the plurality of micropellets remains substantially intact, this indicates that the active agent within at least about 70% of the plurality of micropellets within the dosage form is not exposed to the acidic environment upon ingestion of the dosage by a subject or upon submersion of the dosage form in a solution which simulates gastric fluid. An enteric coating which remains substantially intact may also reflect that at least about 75%, 80%, 85%, 90% or 95% of the active agent within each micropellet is not exposed to the acidic environment upon ingestion or other exposure to acidic media.

The intactness of the enteric coating may be determined by measuring, for example, the degradation of the drug encased within the micropellets. As a specific example, an ER tablet manufactured as described herein, may be incubated, as in standard USP dissolution testing, in a simulated gastric fluid, allowing elution of the micropellets from the dosage form, The micropellets or particles can subsequently be incubated in a media of higher pH in which the enteric coating dissolves and releases the active agent. The amount of drug degradation can be measured by standard methods well known to one skilled in the art, for example, by using an HPLC method to quantitate unchanged drug as well as degradation products, and the results compared to those obtained for control drug samples incubated under the same conditions without enteric coatings.

### IV. Active Agents

The active agent in the dosage form comprise small molecules, peptides, peptide analogs, and peptide mimetics.

One class of active agents for use in the dosage forms are motilin receptor modulators including both agonists and antagonists, including but not limited to, motilin, motilin analogs, and macrolide compounds including, erythromycin and its derivatives, such as rnitemcinal, that are agonists of the motilin receptor. Motilin is a peptide of 22 amino acids which is produced in the gastrointestinal system of a number of species. Motilin induces smooth muscle contractions in the stomach and increases gastric emptying.

Another class of active agents useful in the dosage forms are 5-HT4 receptor modulators, including but not limited to, metoclopramide, mosapride, cisapride, norcisapride, cinitapride, dazopride, prucalopride, renzapride, zacopride, and other benzamide derivatives, and tegaserod. These 5-HT4 agonists and antagonists modulate the ability of 5-hydroxytryptamine (5-HT, i.e. serotonin) to stimulate gut motility. An example is TD-5108 (Theravance), being developed for treatment of disorders with reduced GI motility such as chronic constipation, or TD-8954.

Dopamine receptor antagonists are another class of active agents useful in the dosage forms, including, but not limited to alizapride, bromopride, clebopride, itopride, and doperidome. These drugs possess GI prokinetic activity as well as antiemetic activity.

Yet another class of active agents for use in the dosage forms are growth hormone secretagogue receptor modulators including agonists and antagonists, including but not limited to growth hormone-releasing peptides (GHRPs), also referred to as growth hormone secretagogues (GHS), such as GHRP-1, GHRP-2, GHRP-6, hexarelin, ghrelin, and ghrelin analogs, lanreotide, octreotide, vapreotide, coristatin and coristatin analogs, and macrocyclic ghrelin modulators. Ghrelin is a 28 amino acid peptide which has been shown to improve GI motility in conditions associated with reduced or restricted motility. Examples of ghrelin receptor agonists are TZP-102 (Tranzyme) and RM-131 (Rhythm), each being developed for the treatment of gastroparesis.

Also contemplated for use as the active agents of the dosage forms are acetylcholinesterase inhibitors, muscarinic receptor agonists, and cholecystokinin receptor antagonists which stimulate gastric motility, including but not limited to, pyridostigmine, physostigmine, neostigmine, acotiamide, bethanechol, loxiglumide, and dexloxigluniide.

In one embodiment, the tablet comprises a total of about 1 mg to about 1000 mg, or about 1 mg to about 50 mg, or about 10 mg to about 100 mg, or about 300 mg to about 750 mg, or about 50 mg to about 300 mg of the active agent. In another embodiment, the tablet comprises about 1 mg to 10 mg,5 mg to 40 mg, 20 mg to 80 mg, 50 mg to 200 mg, 100 mg to 300 mg, 300 mg to 850 mg, 350 mg to 800 mg, or 400 mg to 700 mg of the active agent. In still another embodiment, the tablet comprises about 5 mg, 10 mg, 20 mg, 50 mg, 100 mg, 150 mg, 200 mg, 250 mg, 300 mg, 450 mg, 550 mg, 600 mg, 650 mg, 700 mg, 750 mg, 800 mg, 850 mg, or 900 mg of the active agent.

In one embodiment, the tablet comprises about 0.1 wt % to 20% wt %, 0.5 wt % to 10 wt %, 0.1 wt % to 5 wt %, 1 wt % to 15 wt %, 2 wt % to 20 wt %, 5 wt % to 25 wt %, 15 wt % to 75 wt %, 20 wt % to 60 wt %, 30 wt % to 50 wt %, 25 wt % to 50 wt %, 20 wt % to 40 wt %, 15 wt % to 30 wt %, 20 wt % to 30 wt % or 30 wt % to 40 wt % of the active agent. In yet another embodiment, the tablet comprises about 15 wt % (weight percent), or about 50 wt % or about 75 wt % of the active agent. In still another embodiment, the tablet comprises about 0.5 wt %, 1 wt %, 2 wt %, 3 wt %, 4 wt %, 5 wt %, 10 wt %, 15 wt %, 20wt %, 25 wt %, 30 wt %, 35 wt %, 40 wt %, 45 wt %, 55 wt %, 60 wt %, 65 wt %, 70 wt %, 75 wt % or about 80 wt % of the active agent.

In one embodiment, the active agent is a peptide or peptide analog. The peptide active agent comprises from about 2 amino acids to 200 amino acids, 5 amino acids to 150 amino acids, 15 amino acids to 100 amino acids, 50 amino acids to 180 amino acids, 20 amino acids to 120 amino acids, 3 amino acids to 25 amino acids, 15 amino acids to 40 amino acids, 10 amino acids to 60 amino acids, 7 amino acids to 30 amino acids, 4 amino acids to 50 amino acids, or about 20 amino acids to 60 amino acids. In another embodiment, the peptide active agent comprises about 3 amino acids to 10 amino acids, 5 amino acids to 15 amino acids, 10 amino acids to 20 amino acids, 15 amino acids to 30 amino acids, 20 amino acids to 40 amino acids, 25 amino acids to 60 amino acids, 30 amino acids to 75 amino acids, or about 40 amino acids to 90 amino acids. In still another embodiment, the peptide active agent comprises about 5 amino acids, 8 amino acids, 10 amino acids, 12 amino acids, 15 amino acids, 18 amino acids, 22 amino acids, 26 amino acids, 30 amino acids, 35 amino acids, 40 amino acids, or 50 amino acids.

### V. Additional Active Agents

In some embodiments, additional active agents may be administered with the first active agent, either separately or together. Additional active agents from a variety of drug classes including, but not limited to, analgesics, steroids, anti-inflammatory agents, antibiotics, opioids, proton pump inhibitors, antacids, histamine H2 receptor antagonists, and cytokine modulators, may be administered concurrently with active agent, depending on which disorder is being treated.

Suitable anti-inflammatory compounds include, but are not limited to: non-steroidal anti-inflammatory drugs (NSAIDs) (e.g., aspirin, ibuprofen, naproxen, methyl salicylate, diflunisal, indomethacin, sulindac, diclofenac, ketoprofen, ketorolac, carprofen, fenoprofen, flurbiprofen, mefenamic acid, piroxicam, meloxicam, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, triamcinolone, and fluticasone), anti-malarial agents (e.g., hydroxychloroquine), acetaminophen, glucocorticoids, steroids, beta-agonists, anticholinergic agents, methyl xanthines, gold injections, sulphasalazine, penicillamine, anngiogenic agents, dapsone, psoralens, anti-viral agents, and antibiotics. Suitable analgesics include, but are not limited to, NSAIDS, GABA analogs such as baclofen for relief of pain from spasticity, acetaminophen, and opioids.

Opioids useful in the dosage forms and methods include adulmine, alfentanil, allocryptopine, allylprodine, alphaprodine, anileridine, aporphine, benzylmorphine, berberine, bicuculine, bicucine,bezitramide, buprenorphine, bulbocaprine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, elhylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tapentadol, tilidine, and tramadol.

Proton pump inhibitors useful in the dosage forms and methods include omeprazole, esomeprazole, lansoprazole, pantoprazole and rabeprazole sodium.

Suitable histamine H2 receptor antagonists include ranitidine, cimetidine, nizatidine, famotidine, anitidinem sufotidine, roxatidine, bisfentidine, tiotidine, lamtidine, niperotidine, niifentidine, zaltidine, and loxtidine.

### VI. Dosage Forms of Additional Agents

For those embodiments that include further administering additional therapeutic agents simultaneously with active agent, one or more additional agents can be formulated as part of the ER dosage form that includes the first active agent or the one or more additional agents can be administered using one or more dosage forms that are separate from the first active agent. Such dosage forms can be any suitable formulation as are well known in the art. Exemplary dosage forms are described below.

For those additional agents where modified release is desirable, the agent may be incorporated in the active agent ER dosage form or be administered in a separate extended or other modified release formulation dosage form. For those additional agents where immediate release is desirable, the agent may be incorporated in a coating around the active agent ER dosage form or in a separate layer of a bilayer tablet, the agent may be simply enclosed in the capsule of the aforementioned active agent ER capsule dosage form, or the agent may be administered in a separate immediate release dosage form.

Typically, dosage forms contain the additional agent in combination with one or more pharmaceutically acceptable ingredients. The carrier may be in the form of a solid, semi-solid or liquid diluent, or a capsule. Usually the amount of active agent is about 0.1 wt % to 95 wt %, more typically about 1 wt % to 50 wt %. Actual methods of preparing such dosage forms are known, or will be apparent, to those skilled in this art; for example, see Remin ton's Pharmaceutical Sciences, Mack Publishing Company, Easton, Pa., 18th Edition, 1990. The dosage form to be administered will, in any event, contain a quantity of the additional therapeutic agent(s) in an amount effective to alleviate the symptoms of the subject being treated.

In the preparation of pharmaceutical formulations containing the additional therapeutic agent in the form of dosage units for oral administration the agent may be mixed with solid, powdered ingredients, such as lactose, microcrystalline cellulose, maltodextrin, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl funiarate and polyethylene glycol waxes. The mixture is then processed into granules or pressed into tablets such as chewable and oral disintegrating tablets.

Soft gelatin capsules may be prepared by mixing the active agent and vegetable oil, fat, or other suitable vehicle. Hard gelatin capsules may contain granules of the active agent, alone or in combination with solid powdered ingredients such as lactose, saccharose, sorbitol, mannitol, potato starch, corn starch, amylopectin, cellulose derivatives or gelatin.

Liquid preparations for oral administration may be prepared in the form of syrups or suspensions, e.g. solutions or suspensions containing about 0.2-20 wt % of the active agent and the remainder consisting of sugar or sugar alcohols and a mixture of ethanol, water, glycerol, propylene glycol and polyethylene glycol. If desired, such liquid preparations may contain coloring agents, flavoring agents, saccharin and carboxymethyl cellulose or other thickening agents. Liquid preparations for oral administration may also be prepared in the form of a dry powder to be reconstituted with a suitable solvent prior to use.

In certain embodiments, the additional active agent is incorporated in the ER dosage form containing the first active agent. In one embodiment, the second therapeutic agent is formulated for extended release or immediate release. In another embodiment, the dosage form is formulated to provide sustained release of the active agent and the second therapeutic agent.

The amount of the additional active agent in the dosage form can vary. In one embodiment, the composition may comprise from about 1.0 mg to about 1500 mg of the second active agent. In another embodiment, the amount of second active agent in the composition may range from about 100 mg to about 1000 mg. In another embodiment, the amount of second active agent in the composition may range Thorn about 50 mg to about 500 mg. In another embodiment, the amount of second active agent in the composition may range from about 10 mg to about 100 mg. In yet another embodiment, the amount of second active agent in the composition may range from about 1.0 mg to about 10 mg. In one embodiment, the amount of second active agent in the composition may range from about 250 mg to about 1300 mg. In another embodiment, the amount of second active agent in the composition may range from about 325 mg to about 650 mg. In still another embodiment, the amount of second active agent in the composition may range from about 650 mg to about 1300 mg. In one embodiment, only the ER portion of the dosage form contains the second active agent. In another embodiment, only the IR portion of the dosage form contains the second active agent. In yet another embodiment, the second active agent is present in both the IR and ER portions of the dosage form.

### Methods of Making and Characterizing the ER Dosage Forms

Another aspect of the disclosure provides methods for preparing solid dosage forms of the ER pharmaceutical composition that provides extended release of active agent. Solid dosage pharmaceutical compositions in the form of tablets may be produced using any suitable method known in the art including but not limited to wet granulation, dry granulation, direct compression, and combinations thereof.

In one embodiment, a method of making a gastric retentive ER dosage form comprising an active agent and at least one hydrophilic polymer is provided. Gastric retentive dosage forms containing from 0.1 mg to 900 mg of active agent can be manufactured using standard granulation techniques. Tablets are typically prepared using hydroxypropylmethylcellulose (HPMC, Methocel®), polyethylene oxide (PolyOx®), microcrystalline cellulose (Avicel® PH-101), and a suitable lubricant, e.g., magnesium stearate. The dry blend can be compressed into tablets. In one embodiment, a 300 mg active agent tablet contains about 45 wt % active agent, about 16.5 wt % Methocel® K4M, about 3 wt % Methocel® E5, about 22 wt % PolyOx® WSR 303, about 13 wt % Avicel® PH-101, and 1 wt % magnesium stearate, for a total tablet weight of about 670 mg.

The in vitro release of active agent from the tablets can be measured in a USP type 1 apparatus, at 100 rpm, in water or modified simulated gastric fluid. The release of active agent at various time points is measured, for example at 1 hour (h), 2 h, 4 h, 6 h, 8 h, and 10 h, and values of about 20%, 32%, 50%, 63%, 74%, and 83% active agent (cumulative) are obtained.

The in vivo release of active agent from tablets can be determined by measuring the concentration of active agent in plasma samples drawn from subjects at various time points after administration of the treatment. For a single dose of 600 mg active agent gastric retained ER tablets (two 300 mg tablets), a Tmax of about 6 hours, a Cmax of about 25 ug/mL, and an AUC_{inf} of about 43 ug/mL-hour is obtained. These values demonstrate sustained release of active agent with a lower Cmax and longer

Tmax compared to the same dose of active agent in an immediate release dosage form, without loss of bioavailability as measured by the plasma AUC_{inf}.

### EXAMPLES

The following examples are put forth so as to provide those of ordinary skill in the art with a complete disclosure and description of how to make and use the present dosage forms, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., pL, picoliter(s); s or sec, second(s); min, minute(s); h or hr, hour(s); intramuscular(ly); i.p., intraperitoneal(ly); s.c., subcutaneous(ly); and the like.

### Example 1

### Gastric Retentive RM-131 Dosage Forms

Four formulations of gastric retentive tablets of RM-131 are fabricated (2 with 10 pg and 2 with 100 pg RM-131). To ensure that the active agent will be delivered to the upper GI tract, the period of 80% drug release is designed to be approximately 4 to 8 hours. Since retention and drug release represent a balance between swelling and erosion, respectively, two tablet designs are made. One formulation involves conventional tableting to produce a single layer tablet. The other, a bilayer tablet, swells to a greater extent to ensure retention, but is more difficult to manufacture. Each tablet contains 300 mg microcrystalline cellulose. The small blender is lined with a smaller polyethylene bag to reduce the volume to minimize surface adsorption of the drug, and the bag is then coated with 5% of the total microcrystalline cellulose (MCC) by mixing for 2 minutes. The total drug content is then added to the bag in the blender with a remaining 5% of the microcrystalline cellulose to sandwich the drug and mixed for 5 minutes. Except for 10% of the total MCC, the remainder of the MCC is added to the blender in up to 2 separate procedures as geometric blending, and blended for 5 minutes. The bag is removed from the blender and the last portion of the MCC is added to the blender to coat by mixing for 2 minutes. An alternative process to obtain acceptable content uniformity with this low dose is to spray the drug in an ethanolic solution on the total MCC content in a fluid bed and then dry the granulation. The remainder of the drug-containing layer or tablet formulation, that is, 400 mg of polyethylene oxide, the rate-controlling polymer, PolyOx WSR N-60K, and the lubricant 7 mg magnesium stearate, are added to the blender followed by the drug-containing blend and mixed for 10 minutes. Separate blends are prepared for each of the 2 drug contents (10 ug and 100 ug). The single layer tablets with each drug content blend are then tableted by hand on an Auto C Press (Fred Carver, Inc., Indiana) and compressed into approximately 707 mg tablets using a 0.3937″×0.7086″ Modified Oval die (Natoli Engineering, St. Charles, Mo.). The parameters for the operation of the carver Auto C Press are as follows: 3000 lbs force, 0 second dwell time (the setting on the Carver Press), and 100% pump speed.

For the bilayer tablets, a second blend is prepared to form the swelling layer by mixing for 5 minutes in a small blender for each tablet 300 mg polyethylene oxide (PolyOx 303), and 5 mg magnesium stearate as lubricant. After compressing the first layer, the second layer containing 303 mg is added to the tooling, and the full 1010 mg (approximately) tablets for each drug content are compressed.

### Example

### 2

### Gastric Retentive RM-131 Dosage Forms with Varying Release Rates

To vary the release rate, 707 mg single layer tablets of 50 pg RM131 are prepared by substituting the following different polyethylene oxides contents per tablet as the release controlling polymers: a) as above 400 mg POLYOX WSR N60-K, b) 250 mg POLYOX WSR N--60K, c) 400 mg POLYOX 1105, and d) 250 mg POLYOX 1105. The MCC content is adjusted so as to give a combined total of MCC plus polyethylene oxide of 700 mg.

### Example 3

### ER Dosage Form Containing 400 mg Azithromycin

Tablets containing 400 mg of azithromycin to be given once daily with the evening meal for treating diabetic gastroparesis are prepared. Each tablet also contains 400 mg POLYOX N-60K as the rate-controlling polymer, optionally 8 mg polyvinylpyrrolidone vinyl acetate copolymer (PVP) as a binder, and 8 mg magnesium stearate as a lubricant. Tablets are prepared by direct compression or by fluid bed granulation using the PVP as a binder. Tablets are compressed and handmade on the Carver press as described above.

### Example 4

ER Dosage Form Containing 20 mg Azithromycin

Tablets containing 20 mg of azithromycin are prepared as described in Example 3 above except that 300 mg of MCC is added as filler to replace the higher dose of drug in part. PVP is not included, and direct compression is used for these tablets.

### Example 5

### ER Dosage Forms of TZP-102

Tablets are prepared as described in Example 4 except that TZP-102 is used instead of azithromycin. Two doses of TZP-102 are prepared (10 mg and 40 mg) and for both doses the remainder of the formulation is identical to Example 4.

While the dosage forms and methods have been described with reference to the specific embodiments thereof, it should be understood by those skilled in the art that various changes may be made and equivalents may be substituted without departing from the true spirit and scope. In addition, many modifications may be made to adapt a particular situation, material, composition of matter, process, process step or steps, to the objective, spirit and scope. All such modifications are intended to be within the scope of the claims appended hereto.

1. An extended release oral dosage form comprising a therapeutically effective amount of an active agent for the treatment of a gastric motility disorder.

2. The dosage form of claim 1, where the active agent is selected from the group consisting of 5-HT4 receptor modulators, dopamine receptor antagonists, growth hormone secretagogue receptor modulators, and motilin receptor modulators.

3. The dosage form of claim 1, wherein the extended release dosage form comprises a gastric retained dosage form.

4. The dosage form of claim 1, wherein the extended release dosage form comprises an osmotic pump.

5. The dosage form of claim 1, which further comprises a buffering agent.

6. The dosage form of claim 1, which further comprises an antioxidant.

7. The dosage form of claim 1, wherein the active agent is contained in the dosage form as enteric-coated particles or enteric-coated micropellets.

8. The dosage form of claim 1, wherein the active agent is a growth hormone secretagogue agonist.

9. An extended release oral dosage form comprising a therapeutically effective amount of an active agent which promotes gastric emptying, wherein the dosage form remains in the stomach of a patient for about 2 to about 10 hours, and provides extended release of the active agent.

10. The dosage form of claim 9, wherein the active agent is selected from the group consisting of 5-HT4 receptor modulators, dopamine receptor antagonists, growth hormone secretagogue receptor modulators, and motilin receptor modulators.

11. A method of treating a gastric motility disorder comprising providing the dosage form of claim 1 for administration to a subject in need of treatment. 12, The method according to claim 11, wherein the gastric motility disorder is gastroparesis.
